(19) United States
(12) Patent Application Publication
(10) Pub. No.: US 2024/0123246 A1
(43) Pub. Date: Apr. 18, 2024

(54) METHOD AND APPARATUS FOR DELIVERING ALTERNATING ELECTRIC FIELDS TO A TARGET TISSUE

(71) Applicant: Novocure GmbH, Root D4 (CH)

(72) Inventor: Ori FARBER, Haifa (IL)

(73) Assignee: Novocure GmbH, Root D4 (CH)

(21) Appl. No.: 18/379,504

(22) Filed: Oct. 12, 2023

Related U.S. Application Data
(60) Provisional application No. 63/416,152, filed on Oct. 14, 2022.

(51) Int. Cl. A61N 1/40; G16H 40/63; G16H 50/50

(52) U.S. Cl. CPC A61N 1/40; G16H 40/63; G16H 50/50

(57) ABSTRACT

A computer-implemented method to determine locations of transducers to apply tumor treating fields to a target tissue of a subject's body, the computer-implemented method comprises: obtaining a three-dimensional model of at least a portion of the subject's body, wherein the portion of the subject's body includes the target tissue, wherein a single lung of the subject's body includes the target tissue; determining a first location on the three-dimensional model to place a first transducer, wherein the first location is a front of a thorax of the subject's body; determining a second location on the three-dimensional model to place a second transducer, wherein the second location is a back of the thorax of the subject's body, wherein the single lung of the subject is located between the first transducer and the second transducer; determining a third location on the three-dimensional model to place a third transducer, wherein the third location is on a torso of the subject's body; determining a fourth location on the three-dimensional model to place a fourth transducer, wherein the fourth location is on the torso of the subject's body; and outputting a representation of the first, second, third, and fourth locations on the subject's body.

### CROSS-REFERENCE TO RELATED APPLICATIONS

This application claims priority to U.S. Provisional Application No. 63/416,152, filed Oct. 14, 2022, the contents of which are incorporated by reference herein in their entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into the region of interest by transducers placed on the patient's body and applying alternativing current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1 illustrates a flowchart depicting an example computer-implemented method for determining locations of transducers to apply TTFields to a target tissue of a subject's body.

FIG. 2 illustrates a flowchart depicting an example method of applying TTFields to a subject's body.

FIGS. 3A-3C depict two example transducer layouts for applying TTFields to a subject's body.

FIGS. 4A-4C depict three example transducer layouts for applying TTFields to a subject's body for targeting a single lung.

FIGS. 5A-5E depict examples of mean field intensities in the lungs resulting from the different transducer layouts.

FIG. 6 depicts an example apparatus to apply alternating electric fields to a subject's body.

FIGS. 7A-7B illustrate schematic views of exemplary design of a transducer for applying alternating electric fields.

FIG. 8 depicts an example computer apparatus.

### DESCRIPTION OF EMBODIMENTS

This application describes exemplary methods and apparatuses to determine locations of transducers to apply alternating electric fields (e.g., tumor treating fields (TTFields)) to a target tissue of a subject's body and to apply an alternating electric field to a subject's body.

As realized by the inventor, in administering TTFields treatment to a subject, a goal may be to maximize the energy focused on the tumor, and subjects with lung cancer may have a tumor in both lungs or in a single lung. In addition, a subject with lung cancer, or other another cancer, may have a chemo port, a shunt, a sensitive scar area (e.g., from surgery or radiation treatment), or an anatomic part to avoid (e.g., an ear or a nipple), and in administering TTFields treatment to a subject, a goal may be to avoid such areas when locating transducers on the subject. In considering how to solve these problems, the inventor discovered that particular layouts of transducers for administering TTFields may be more beneficial for subjects having a tumor in a single lung, instead of both lungs, and/or having certain areas to avoid when placing transducers on the subject. The inventor has discovered that placing at least two pairs of transducers at particular locations on the subject's body may provide increased TTFields energy to a target tissue (e.g., tumor). As an example, if a subject has a tumor in one lung, instead of both lungs, the invention may be used to focus the TTFields on the one lung, thereby providing increased TTFields energy to the tumor. Moreover, the inventor has discover that by determining the impact of different transducers and their locations using computer simulations, avoiding certain areas of the subject may be part of the TTFields treatment planning for the subject.

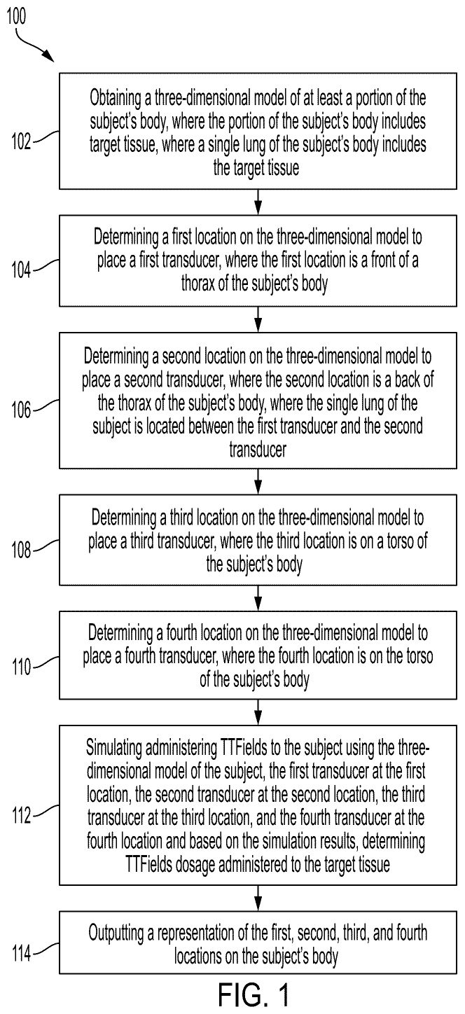

FIG. 1 illustrates a flowchart depicting an example computer-implemented method 100 for determining locations of transducers to apply TTFields to a target tissue of a subject's body. Certain steps of the method 100 are described as computer-implemented steps. The computer may be, for example, any device comprising one or more processors and memory accessible by the one or more processors, the memory storing instructions that when executed by the one or more processors cause the computer to perform the relevant steps of the method 100. The method 100 may be implemented by any suitable system or apparatus, such as the system of FIG. 8. While an order of operations is indicated in FIG. 1 for illustrative purposes, the timing and ordering of such operations may vary where appropriate without negating the purpose and advantages of the examples set forth in detail herein.

With reference to FIG. 1, at step 102, the method 100 may include obtaining a three-dimensional model of at least a portion of the subject's body. The three-dimensional model can be obtained from computer memory locally or over a network. The three-dimensional model may be generated based on one or more images of a region of interest of the subject. In some embodiments, the one or more images are medical images. The medical image may, for example, include at least one of a magnetic resonance imaging (MRI) image, a computerized tomography (CT) image, an X-ray image, an ultrasound image, nuclear medicine image, a positron-emission tomography (PET) image, arthrogram images, myelogram images, or any image of the subject's body providing an internal view of the subject's body. Each medical image may include an outer shape of a portion of the subject's body and a region corresponding to the region of interest (e.g., tumor) within the subject's body. As an example, the medical image may be a three-dimensional (3D) MRI image. In another example, the three-dimensional model may be a computational phantom designed to mimic an actual subject.

In some embodiments, the portion of the subject's body includes the target tissue. The target tissue can include cancer, tumor, lung, brain, or combinations thereof. In some embodiments, a single lung of the subject's body includes the target tissue.

At step 104, the method 100 may include determining a first location on the three-dimensional model to place a first transducer. In some embodiments, the first location may be the front of the thorax of the subject's body. As an example, the thorax of the subject's body may be the region of the subject's body between the neck and the abdomen of the subject. As an example, the thorax of the subject's body may be the cavity of the subject's body containing the heart and the lungs of the subject.

At step 106, the method 100 may include determining a second location on the three-dimensional model to place a second transducer. In some embodiments, the second location may be the back of the thorax of the subject's body.

In some embodiments, a single lung of the subject may be located between the first transducer and the second transducer. In some embodiments, the first and second transducers may form a first pair of transducers for administering TTFields to the subject. In some embodiments, the first and second transducers may be capacitively coupled. In some embodiments, the, the transducers may not be capacitively coupled.

At step 108, the method 100 may include determining a third location on the three-dimensional model to place a third transducer. In some embodiments, the third location may be on the torso of the subject's body. As an example, the torso of the subject's body may be the region of the subject's body from the neck to the groin of the subject. As an example, the torso of the subject's body may be the body of the subject excluding the head and the limbs of the subject.

At step 110, the method 100 may include determining a fourth location on the three-dimensional model to place a fourth transducer. In some embodiments, the fourth location may be on the torso of the subject's body. In some embodiments, the first and second locations may not be overlapping with the third or fourth location.

As an example, the third location may be under a left armpit of the subject, and the fourth location may be under the right armpit of the subject. As an example, the third location may be on the front of the thorax of the subject's body, and the fourth location may be under the armpit of the subject, where the third location is not overlapping with the first location. As an example, the third location may be on the back of the thorax of the subject's body, and the fourth location may be under the armpit of the subject, where the third location is not overlapping with the second location.

The third and fourth transducers may form a second pair of transducers for administering TTFields to the subject. The third and fourth transducers may be capacitively coupled. As another example, the transducers may not be capacitively coupled.

In some embodiments, the first and second transducers in the first pair of transducers may have a same number of electrode elements and may have a same shape, and the third and fourth transducers in the second pair of transducers may have a same number of electrode elements and may have a same shape. The first and second transducers may have at least one of: a different number of electrode elements than the third and fourth transducers, or a different shape than the third and fourth transducers. As an example, the first and second transducers may each have 20 electrode elements, and the third and fourth transducers may each have 13 electrode elements. As an example, the first and second transducers may each have a substantially oval shape, and the third and fourth transducers may each have a substantially circular shape. Other combinations of number of electrode elements and shapes may be used.

The transducers and their locations determined in steps 104, 106, 108, and 110 may be determined automatically and/or based on input by a user. In some embodiments, the one or more transducer placement positions (e.g., the transducers and their locations) may be generated based on, for example, the region of interest of the subject's body corresponding to the target tissue (e.g., a tumor in a single lung). As an example, the one or more transducer placement positions may be intended to optimize the TTFields dose delivered to the region of interest of the subject's body.

At step 112, the method 100 may include simulating administering TTFields to the subject using the three-dimensional model of the subject, the first transducer at the first location, the second transducer at the second location, the third transducer at the third location, and the fourth transducer at the fourth location and based on the simulation results, determining TTFields dosage administered to the target tissue. These calculations in step 112 may involve solving complex algorithms using the large data set of the three-dimensional model of the subject and, as such, require the use of a computer apparatus, as the human mind is not capable of performing the required calculations. If the determined TTFields dosage is sufficient, the transducer layout may be recommended for use on the subject and may be provided as output in step 114.

In some embodiments, with at least one of the third and fourth transducers having a smaller size and/or a different shape than the first and second transducers, certain areas of the subject may be avoided when placing the third and fourth transducers on the subject, such as avoiding a chemo port, a shunt, a sensitive scar area (e.g., from surgery or radiation treatment), or an anatomic part to avoid (e.g., an ear or a nipple) of the subject. As such, by determining the impact of different transducers and their locations using the method 100, avoiding certain areas of the subject may be part of the TTFields treatment planning for the subject.

At step 114, the method 100 may include outputting a representation of the first, second, third, and fourth locations and/or transducers on the subject's body. As an example, a display may be used to show the representation of the first, second, third, and fourth locations and/or transducers on the subject's body. As an example, a document may be used to show the representation of the first, second, third, and fourth locations and/or transducers on the subject's body.

When the transducers are positioned based on the locations determined according to the method 100, the transducers may induce alternating electric fields (e.g., TTFields). As an example, the first and second transducers may generate a first alternating electric field, and the third and fourth transducers may generate a second alternating electric field. In some embodiments, when the first and second alternating electric fields are induced, the single lung of the subject with the tumor may have a higher average electric field intensity than the other lung of the subject.

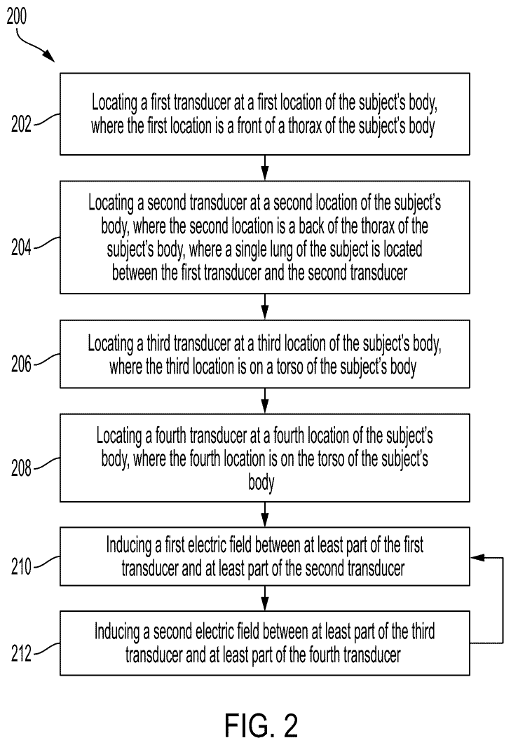

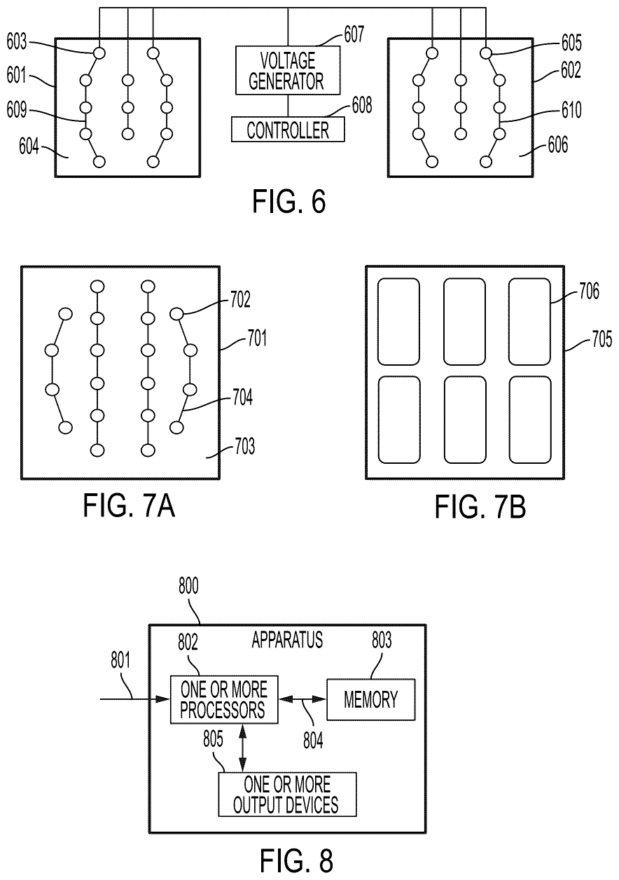

FIG. 2 illustrates a flowchart depicting an example method 200 of applying TTFields to a subject's body. Certain steps of the method 200 are described as computer-implemented steps. The computer may be, for example, any device comprising one or more processors and memory accessible by the one or more processors, the memory storing instructions that when executed by the one or more processors cause the computer to perform the relevant steps of the method 100. The method 200 may be implemented by any suitable system or apparatus, such as the system of FIG. 6 and/or the apparatus of FIG. 8. While an order of operations is indicated in FIG. 2 for illustrative purposes, the timing and ordering of such operations may vary where appropriate without negating the purpose and advantages of the examples set forth in detail herein.

With reference to FIG. 2, at step 202, the method 200 may include locating a first transducer at a first location of the subject's body. In some embodiments, the first location may be the front of the thorax of the subject's body.

At step 204, the method 200 may include locating a second transducer at a second location of the subject's body. In some embodiments, the second location may be the back of the thorax of the subject's body. In some embodiments, a single lung of the subject may be located between the first transducer and the second transducer.

At step 206, the method 200 may include locating a third transducer at a third location of the subject's body. In some embodiments, the third location may be on the torso of the subject's body.

At step 208, the method 200 may include locating a fourth transducer at a fourth location of the subject's body. In some embodiments, the fourth location may be on the torso of the subject's body.

As an example, the third location may be under a left armpit of the subject, and the fourth location may be under the right armpit of the subject. As an example, the third location may be on the front of the thorax of the subject's body, and the fourth location may be under the armpit of the subject, where the third location is not overlapping with the first location. As an example, the third location may be on the back of the thorax of the subject's body, and the fourth location may be under the armpit of the subject, where the third location is not overlapping with the second location.

At step 210, the method 200 may include inducing a first electric field between at least part of the first transducer and at least part of the second transducer by applying an AC voltage between this first pair of transducers.

At step 212, the method 200 may include inducing a second electric field between at least part of the third transducer and at least part of the fourth transducer by applying an AC voltage between this first pair of transducers. Flow cycles between steps 210 and 212 to generate alternating electric fields at a particular interval for a particular period of time depending on a determined TTFields dosage.

As an example, an alternating electric field (e.g., TTFields) may be applied to target tissue (e.g., tumor or cancer in a lung), cells, or the area of a subject. In some embodiments, the alternating electric field may be applied with predetermined parameters. As an example, the alternating electric field may include a frequency within a frequency range from about 50 kHz to about 1 MHz. As an example, the alternating electric field may include a frequency within a frequency range from about 50 kHz to about 10,000 kHz. As an example, the frequency of the alternating electric field may be between approximately 50 kHz and approximately 1000 kHz or between approximately 100 kHz and approximately 300 kHz. As an example, the frequency of the alternating electric field may be approximately 100 kHz, approximately 150 kHz, approximately 200 kHz, approximately 250 kHz, or approximately 300 kHz.

As an example, the alternating electric fields (e.g., TTFields) may include an intensity within a range from about 1 V/cm to about 10 V/cm. As an example, the intensity of the alternating electric field may be between approximately 1 V/cm and approximately 4 V/cm. Other possible exemplary parameters for the alternating electric field may include active time, dimming time, and duty cycle (all of which may be measured in, for example, ms units), among other parameters. The parameters may be modified based on the conditions of the subject (e.g., the sizes of the target tissue, types of tumor, age, or sex of the subject) or the purposes of the treatment. As an example, the intensity of the alternating electric field may be between approximately 1 V/cm and approximately 4 V/cm, and the frequency of the alternating electric field may be between approximately 150 kHz and approximately 250 kHz for treating tumor/cancer cells. In some embodiments, the alternating electric field may be applied using two pairs of transducer arrays placed on the subject and directed on a target tissue (e.g., tumor) of the subject.

In some embodiments, when the first and second electric fields are induced, a single lung may of the subject have a higher average electric field intensity than the other lung of the subject. In some embodiments, the single lung may have an average electric field intensity of at least 1.0 V/cm when the alternating electric field is applied.

Various combinations of pairs of transducers, as discussed herein, or similar pairs of transducers may be used together. Various locations of transducers, such as those discussed herein or in other locations, may be used. A transducer may be used in a single pair of transducers or in two or more pairs of transducers. A transducer may be partitioned to be used in a single pair of transducers or in two or more pairs of transducers. The transducers, the transducer locations, the pairs of transducers, and the two or more pairs of transducers discussed herein are not exhaustive.

### EXPERIMENTAL RESULTS

TTFields may be considered to be an anti-mitotic novel therapy utilizing low-intensity alternating electric fields to arrest cell proliferation. TTFields may be delivered using two pairs of transducers placed on the subject's skin. The distribution of the fields is determined by the electric properties of the tissues and by the geometry of the subject and the system. Thus, the location of the transducers on the subject's body may have a significant influence on the TTFields dose the subject may receive. A transducer layout may be prescribed according to clinical guidelines for thoracic disease, taking into account the size of the patient. However, the actual placement of the transducers may pose challenges. In many cases, lung cancer patients also have shunts that may overlap the location of the arrays. In addition, areas with skin damage due to surgery or radiation therapy may prevent placing the arrays on these locations, ultimately resulting in different field distributions than expected. Accordingly, as discovered by the inventor, exemplary methods and apparatuses may be used to determine locations of transducers to apply alternating electric fields to a target tissue of a subject's body and to apply an alternating electric field to a subject's body to treat one or more cancers/tumors located in the subject's body.

Using Sim4Life V6.2 (ZMT Zurich), the electric field distribution was simulated within a healthy human model (Duke, by ITI'S foundation, Zurich). Dirichlet boundary conditions were applied on surfaces of different sizes (25% to 121% of the area of the actual transducers used for the thorax) adhered to the model's skin. The surfaces were positioned in different locations: on top of the clavicle bone, on top of the heart, and on the bottom of the lung such that the bottom edge coincides with the diaphragm. Then the field distribution in the lungs resulting from each surface placement was analyzed and compared to the distribution from a surface roughly at the size of the real-world transducers. In addition, the resistance of the model was analyzed in each case since this has direct implications for actual current delivery during treatment.

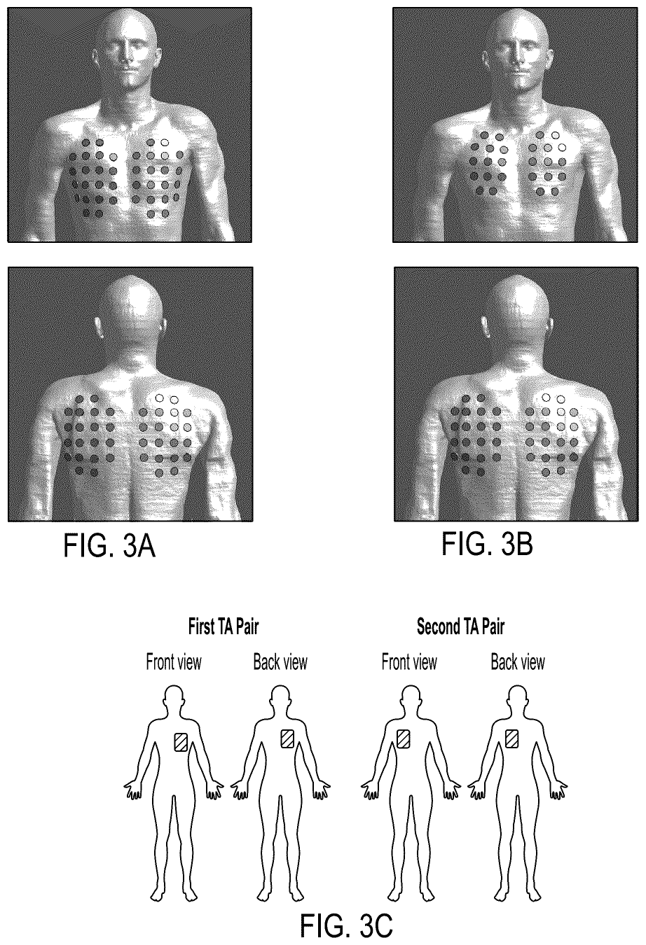

Delivery of TTFields to the lung was evaluated by placing arrays consisting of 13 or 20 ceramic discs on the skin of a realistic human computational phantom. Sim4Life v6.0 software was then used to simulate TTFields distribution. The distribution of TTFields was assessed for five layouts. First, FIGS. 3A-3C depict two example transducer layouts for applying TTFields to a subject's body. For two guideline-recommended layouts (FIGS. 3A-3C), four arrays were placed on the front and back of the thorax and coupled in a cross formation (i.e., back left with front right and vice versa), creating approximately diagonal fields within the body. FIGS. 3A-3C show transuder layouts used for treating lung cancers in male patients. FIG. 3A shows a first cross transducer layout (both pairs have 20-disc transducer arrays), and FIG. 3B shows a second cross transducer layout (each pair has a 20-disc transducer array on the back and a 13-disc transducer array on the front). FIG. 3C shows a front view and a back view of the two transducer locations (e.g., the first transducer pair and the second transducer pair).

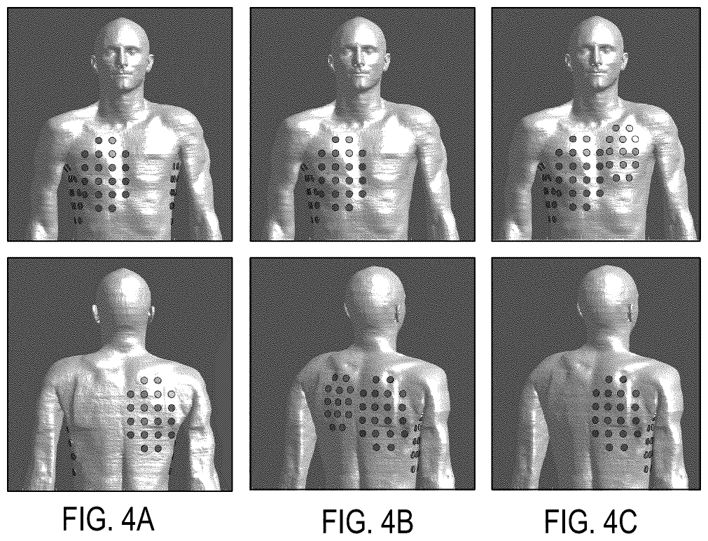

Second, FIGS. 4A-4C depict three example transducer layouts for applying TTFields to a subject's body for targeting a single lung. According to some embodiments, for three single lung transducer layouts (FIGS. 4A-4C), a channel was focused on the right lung (front and back lateral channel) and was paired with a channel in which two transducers were placed under each armpit or where a transducer was placed at the front or back of the left lung and another transducer was placed under the right armpit. A current of 4.0 A was set for channels having the 20-disc transducer arrays, while a current of 2.6 A was set for channels having at least one 13-disc transducer array. The average field intensity delivered to each lung was calculated for each layout. FIGS. 4A-4C show the transducer layouts situated to target the right lung. For this example, each transducer includes two 20-disc transducer arrays placed on top of a single (right) lung. In FIG. 4A, the second channel includes two 13-disc transducer arrays placed under each armpit. In FIG. 4B and FIG. 4C, the second channel includes a first 13-disc transducer array placed under the right armpit and a second 13-disc transducer array placed on the back (FIG. 4B) or front (FIG. 4C) of the left lung.

The lung region with peak field intensities shifted in accordance with the transducer location and, in general, remained around the centerline connecting the transducers. While therapeutic levels may still be reached outside this area, field intensities tended to decrease as the distance from the transducers increases. Analysis of the resistance showed high sensitivity to the size of the transducers, with resistance increasing by up to 2.5-fold from the baseline size. However, the transducer location had little impact on the resistance, with a maximal span of 5Ω over the resistance of 45Ω (observed for the smallest transducers). The vertical location of the area with higher field intensities shifted with the vertical location of the transducers relative to the lungs. This localization effect diminished as the transducers become larger. Nevertheless, even with relatively small transducers, therapeutic levels of TTFields may still be reached throughout most of the lungs regardless of the vertical location of the transducers.

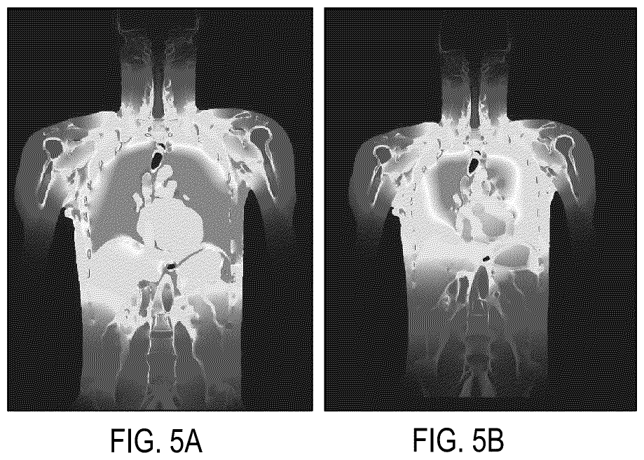

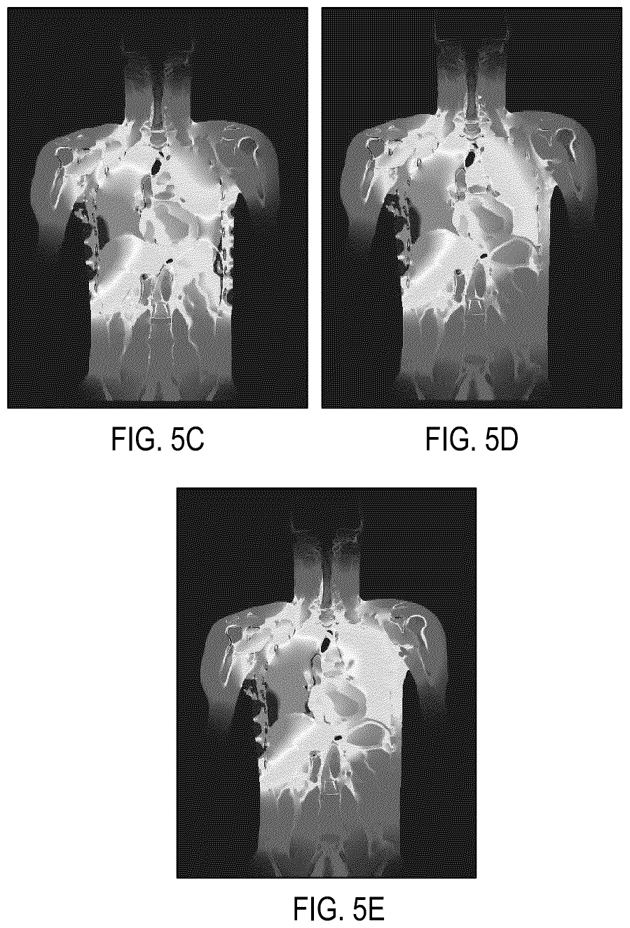

FIGS. 5A-5E depict examples of mean field intensities in the lungs resulting from the different layouts. As can be seen in FIGS. 5A and 5B, which correspond to the transducer layouts in FIGS. 3A and 3B, respectively, these transducer layouts provide better whole-lung coverage for both lungs. In contrast, as can be seen in FIGS. 5C, 5D, and 5E, which correspond to the transducer layouts in FIGS. 4A, 4B, and 4C, respectively, these transducer layouts focused on the the target (right) lung and provide coverage in the lower parts of the right lung with lower values for the upper parts of the right lung and the whole left lung. In terms of local minimum power density (LMiPD), the guideline-recommended transducer layout (FIG. 3A) provided 0.83 mW/cm³ for both lungs, while the inventive transducer layouts (FIGS. 4A, 4B, and 4C) provided 0.62-0.81 mW/cm³ and 0.22-0.27 mW/cm³ to the right and left lungs, respectively.

Table 1 below provides simulation results for the five transducer layouts of FIGS. 3A, 3B, 4A, 4B, and 4C and their respective mean field intensities in FIGS. 5A, 5B, 5C, 5D, and 5E, respectively. For the guideline-recommended transducer layout (FIG. 3A), the average field intensity resulting from the recommended layout was 1.69 V/cm for each lung. The inventive transducer layouts (FIGS. 4A, 4B, and 4C) provided 1.60 V/cm to 1.69 V/cm for the right lung and 0.88 V/cm to 0.99 V/cm for the left lung. The field intensity was locally averaged over the two channels of the each transducer layout to arrive at the values in Table 1.

TABLE 1
Field intensities within each lung
resulting from each transducer layout.

| | | Average | | Volume | |
|---|---|---|---|---|---|
| | | Field | | over | |
| | | Intentsity | | 1 V/cm | |
| | | [V/cm] | | [%] | |
| | Transducer Layout | Left | Right | Left | Right |
| FIGS. | (Number of discs) | Lung | Lung | Lung | Lung |
| 3A, 5A | Cross layout (both 20) | 1.69 | 1.69 | 98.99 | 99.35 |
| 3B, 5B | Cross layout (each pair 20 + 13) | 1.26 | 1.33 | 83.26 | 90.61 |
| 4A, 5C | Target lung (20) and lateral (13) | 0.88 | 1.60 | 28.09 | 95.05 |
| 4B, 5D | Target lung (20) and adjusted | 0.98 | 1.69 | 38.66 | 92.49 |
| | lateral back (13) | | | | |
| 4C, 5E | Target lung (20) and adjusted | 0.99 | 1.67 | 31.37 | 97.44 |
| | lateral front (13) | | | | |

The disclosed results show that the field intensities in the lungs are sensitive to the location of the arrays. The magnitude of this sensitivity depends on the size of the arrays. The need for smaller arrays under the armpits may limit the expected output current from the device. In addition, the location of the smaller arrays may prevent the electric field intensity from sufficiently covering regions surrounding the mediastinum. However, if a tumor is located in a single lung of the subject, some embodiments of the transducer layouts for administering TTFields may be more beneficial for such by focusing the energy of the TTFields on the single lung, instead of both lungs. Further, having smaller transducers may still provide a similar amount of TTFields therapy to the single lung energy but also provide the ability to avoid certain areas (e.g., a chemo port, a shunt, a sensitive scar area (e.g., from surgery or radiation treatment), or an anatomic part to avoid (e.g., an ear or a nipple)) when placing the smaller transducers on the subject.

### Exemplary Apparatuses

FIG. 6 depicts an example apparatus to apply alternating electric fields (e.g., TTFields) to a subject's body. The first transducer array 601 includes 13 electrode elements 603, which are positioned on the substrate 604, and the electrode elements 603 are electrically and mechanically connected to one another through a conductive wiring 609. The second transducer array 602 includes 13 electrode elements 605, which are positioned on the substrate 606, and the electrode elements 605 are electrically and mechanically connected to one another through a conductive wiring 610. The first transducer array 601 and the second transducer array 602 are connected to an AC voltage generator 607 and a controller 608. The controller 608 may include one or more processors and memory accessible by the one or more processors. The memory may store instructions that when executed by the one or more processors, control the AC voltage generator 607 to implement one or more embodiments of the invention. In some embodiments, the AC voltage generator 607 and the controller 608 may be integrated in the first transducer array 601 and the second transducer array 602 and form a first electric field generator and a second electric field generator.

The structure of the transducers may take many forms. The transducers may be affixed to the subject's body or attached to or incorporated in clothing covering the subject's body. The transducer may include suitable materials for attaching the transducer to a subject's body. For example, the suitable materials may include cloth, foam, flexible plastic, and/or a conductive medical gel. The transducer may be conductive or non-conductive.

The transducer may include any desired number of electrode elements. Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In some embodiments, at least one electrode element of the first, the second, the third, or the fourth transducer can include at least one ceramic disk that is adapted to generate an alternating electric field. In non-limiting embodiments, at least one electrode element of the first, the second, the third, or the fourth transducer may include a polymer film that is adapted to generate an alternating field. In some embodiments, the disclosed systems can have more than four transducers.

FIG. 7A illustrates a schematic view of an exemplary design of a transducer for applying alternating electric fields. The transducer array 701 includes 20 electrode elements 702, which are positioned on the substrate 703, and the electrode element 702 are electrically and mechanically connected to one another through a conductive wiring 704. In some embodiments, the electrode elements 702 may include a ceramic disk.

FIG. 7B illustrates a schematic view of an exemplary design of a transducer for applying alternating electric fields. The transducer 705 may include substantially flat electrode elements 706. In some embodiments, the electrode elements 706 are non-ceramic dielectric materials positioned over flat conductors. Examples of non-ceramic dielectric materials positioned over flat conductors may include polymer films disposed over pads on a printed circuit board or over flat pieces of metal. In some embodiments, such polymer films have a high dielectric constant, such as, for example, a dielectric constant greater than 10. In some embodiments, electrode elements 706 may have various shapes. For example, the electrode elements may be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape. In some embodiments, each of electrode elements 706 may have a same shape, similar shapes, and/or different shapes.

FIG. 8 depicts an example computer apparatus for use with the embodiments herein. As an example, the apparatus 800 may be a computer to implement certain inventive techniques disclosed herein. As an example, the methods of FIGS. 1 and 2 may be performed by a computer, such as apparatus 800. As an example, the apparatus 800 may be a controller apparatus to apply the alternating electric fields (e.g., TTFields) according to the embodiments herein. The controller apparatus 800 may be used as the controller 608 of FIG. 6. The apparatus 800 may include one or more processors 802, memory 803, one or more input devices (not shown), and one or more output devices 805.

In some embodiments, based on input 801, the one or more processors 802 may generate control signals to control the voltage generator to implement one or more embodiments of the invention. As an example, the input 801 is user input. As an example, the input 801 may be from another computer in communication with the apparatus 800. The input 801 may be received in conjunction with one or more input devices (not shown) of the apparatus 800.

The memory 803 may be accessible by the one or more processors 802 (e.g., via the link 804) so that the one or more processors 802 can read information from and write information to the memory 803. The memory 803 may store instructions that when executed by the one or more processors 802 implement one or more embodiments described herein. The memory 803 may be a non-transitory computer readable medium (or a non-transitory processor readable medium) containing a set of instructions thereon, where when executed by a processor (such as one or more processors 802), the instructions cause the processor to perform one or more methods disclosed herein.

The one or more output devices 805 may provide the status of the computer-implemented techniques herein. The one or more output devices 805 may provide visualization data according to some embodiments of the invention.

The apparatus 800 may include: one or more processors (such as one or more processors 802); and memory (such as memory 803) accessible by the one or more processors, the memory storing instructions that when executed by the one or more processors, cause the apparatus to perform one or more methods disclosed herein.

### ILLUSTRATIVE EMBODIMENTS

The invention includes other illustrative embodiments, such as the following.
Illustrative Embodiment 1. A computer-implemented method to determine locations of transducers to apply tumor treating fields to a target tissue of a subject's body, the computer-implemented method comprises: obtaining a three-dimensional model of at least a portion of the subject's body, wherein the portion of the subject's body includes the target tissue, wherein a single lung of the subject's body includes the target tissue; determining a first location on the three-dimensional model to place a first transducer, wherein the first location is a front of a thorax of the subject's body; determining a second location on the three-dimensional model to place a second transducer, wherein the second location is a back of the thorax of the subject's body, wherein the single lung of the subject is located between the first transducer and the second transducer; determining a third location on the three-dimensional model to place a third transducer, wherein the third location is on a torso of the subject's body; determining a fourth location on the three-dimensional model to place a fourth transducer, wherein the fourth location is on the torso of the subject's body; and outputting a representation of the first, second, third, and fourth locations on the subject's body.
Illustrative Embodiment 2. The computer-implemented method of Illustrative Embodiment 1, wherein the third location is under a left armpit of the subject, and the fourth location is under the right armpit of the subject.
Illustrative Embodiment 3. The computer-implemented method of Illustrative Embodiment 1, the third location is on the front of the thorax of the subject's body, and the fourth location is under an armpit of the subject, wherein the third location is not overlapping with the first location.
Illustrative Embodiment 4. The computer-implemented method of Illustrative Embodiment 1, wherein the third location is on the back of the thorax of the subject's body, and the fourth location is under an armpit of the subject, wherein the third location is not overlapping with the second location.
Illustrative Embodiment 5. The computer-implemented method of Illustrative Embodiment 1, wherein a first alternating electric field is simulated as being generated by the first transducer at the first location and the second transducer at the second location, wherein a second alternating electric field is simulated as being generated by the third transducer at the third location and the fourth transducer at the fourth location, wherein for the simulated first alternating electric field and the simulated second alternating electric field, the single lung has a higher average electric field intensity than another lung.
Illustrative Embodiment 6. The computer-implemented method of Illustrative Embodiment 5, wherein the single lung has a simulated average electric field intensity of at least 1.0 V/cm.
Illustrative Embodiment 7. The computer-implemented method of Illustrative Embodiment 1, wherein the first and second transducers have a same number of electrode elements and have a same shape, wherein the third and fourth transducers have a same number of electrode elements and have a same shape, wherein the first and second transducers have at least one of: a different number of electrode elements than the third and fourth transducers, or a different shape than the third and fourth transducers.
Illustrative Embodiment 8. A system to apply tumor treating fields to a subject's body, the system comprising: a first transducer adapted to be located at a first location of the subject's body, wherein the first location is a front of a thorax of the subject's body; a second transducer adapted to be located at a second location of the subject's body, wherein the second location is a back of the thorax of the subject's body, wherein a single lung of the subject is located between the first location and the second location; a third transducer adapted to be located at a third location of the subject's body, wherein the third location is on a torso of the subject's body; a fourth transducer adapted to be located at a fourth location of the subject's body, wherein the fourth location is on the torso of the subject's body; a voltage generator adapted to provide a first voltage to the first transducer, a second voltage to the second transducer, a third voltage to the third transducer, and a fourth voltage to the fourth transducer; and a controller coupled to the voltage generator, wherein the controller is adapted to instruct the voltage generator to induce a first alternating electric field between at least part of the first transducer and at least part of the second transducer and a second alternating electric field between at least part of the third transducer and at least part of the fourth transducer.
Illustrative Embodiment 9. The system of Illustrative Embodiment 8, wherein the third location is under a left armpit of the subject, and the fourth location is under the right armpit of the subject.
Illustrative Embodiment 10. The system of Illustrative Embodiment 8, the third location is on the front of the thorax of the subject's body, and the fourth location is under an armpit of the subject, wherein the third location is not overlapping with the first location.
Illustrative Embodiment 11. The system of Illustrative Embodiment 8, wherein the third location is on the back of the thorax of the subject's body, and the fourth location is under an armpit of the subject, wherein the third location is not overlapping with the second location.
Illustrative Embodiment 12. The system of Illustrative Embodiment 8, when the first and second alternating electric fields are induced, the single lung has a higher average electric field intensity than another lung.
Illustrative Embodiment 13. The system of Illustrative Embodiment 8, wherein the at least one electrode element of the first, the second, the third, or the fourth transducer comprises at least one ceramic disk that is adapted to generate an alternating electric field.
Illustrative Embodiment 14. The system of Illustrative Embodiment 8, wherein the at least one electrode element of the first, the second, the third, or the fourth transducer comprises a polymer film that is adapted to generate an alternating field.
Illustrative Embodiment 15. A method of applying tumor treating fields to a subject's body, the method comprising: locating a first transducer at a first location of the subject's body, wherein the first location is a front of a thorax of the subject's body; locating a second transducer at a second location of the subject's body, wherein the second location is a back of the thorax of the subject's body, wherein a single lung of the subject is located between the first transducer and the second transducer; locating a third transducer at a third location of the subject's body, wherein the third location is on a torso of the subject's body; locating a fourth transducer at a fourth location of the subject's body, wherein the fourth location is on the torso of the subject's body; inducing a first alternating electric field between at least part of the first transducer and at least part of the second transducer; and inducing a second alternating electric field between at least part of the third transducer and at least part of the fourth transducer.
Illustrative Embodiment 16. The method of Illustrative Embodiment 15, wherein the third location is under a left armpit of the subject, and the fourth location is under the right armpit of the subject.
Illustrative Embodiment 17. The method of Illustrative Embodiment 15, the third location is on the front of the thorax of the subject's body, and the fourth location is under an armpit of the subject, wherein the third location is not overlapping with the first location.
Illustrative Embodiment 18. The method of Illustrative Embodiment 15, wherein the third location is on the back of the thorax of the subject's body, and the fourth location is under an armpit of the subject, wherein the third location is not overlapping with the second location.
Illustrative Embodiment 19. The method of Illustrative Embodiment 15, when the first and second alternating electric fields are induced, the single lung has a higher average electric field intensity than another lung.
Illustrative Embodiment 20. The method of Illustrative Embodiment 19, wherein the single lung has an average electric field intensity of at least 1.0 V/cm.
Illustrative Embodiment 21. A device, method, and/or system substantially as shown and described.

Embodiments illustrated under any heading or in any portion of the disclosure may be combined with embodiments illustrated under the same or any other heading or other portion of the disclosure unless otherwise indicated herein or otherwise clearly contradicted by context.

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A computer-implemented method to determine locations of transducers to apply tumor treating fields to a target tissue of a subject's body, the computer-implemented method comprising:
obtaining a three-dimensional model of at least a portion of the subject's body, wherein the portion of the subject's body includes the target tissue, wherein a single lung of the subject's body includes the target tissue;
determining a first location on the three-dimensional model to place a first transducer, wherein the first location is a front of a thorax of the subject's body;
determining a second location on the three-dimensional model to place a second transducer, wherein the second location is a back of the thorax of the subject's body, wherein the single lung of the subject is located between the first transducer and the second transducer;
determining a third location on the three-dimensional model to place a third transducer, wherein the third location is on a torso of the subject's body;
determining a fourth location on the three-dimensional model to place a fourth transducer, wherein the fourth location is on the torso of the subject's body; and
outputting a representation of the first, second, third, and fourth locations on the subject's body.

2. The computer-implemented method of claim 1, wherein the third location is under a left armpit of the subject and the fourth location is under the right armpit of the subject.

3. The computer-implemented method of claim 1, the third location is on the front of the thorax of the subject's body, and the fourth location is under an armpit of the subject, wherein the third location is not overlapping with the first location.

4. The computer-implemented method of claim 1, wherein the third location is on the back of the thorax of the subject's body, and the fourth location is under an armpit of the subject, wherein the third location is not overlapping with the second location.

5. The computer-implemented method of claim 1, wherein a first alternating electric field is simulated as being generated by the first transducer at the first location and the second transducer at the second location,
wherein a second alternating electric field is simulated as being generated by the third transducer at the third location and the fourth transducer at the fourth location,
wherein for the simulated first alternating electric field and the simulated second alternating electric field, the single lung has a higher average electric field intensity than another lung.

6. The computer-implemented method of claim 5, wherein the single lung has a simulated average electric field intensity of at least 1.0 V/cm.

7. The computer-implemented method of claim 1, wherein the first and second transducers have a same number of electrode elements and have a same shape,
wherein the third and fourth transducers have a same number of electrode elements and have a same shape,
wherein the first and second transducers have at least one of:
a different number of electrode elements than the third and fourth transducers, or
a different shape than the third and fourth transducers.

8. A system to apply tumor treating fields to a subject's body, the system comprising:
a first transducer adapted to be located at a first location of the subject's body, wherein the first location is a front of a thorax of the subject's body;
a second transducer adapted to be located at a second location of the subject's body, wherein the second location is a back of the thorax of the subject's body, wherein a single lung of the subject is to be located between the first location and the second location;
a third transducer adapted to be located at a third location of the subject's body, wherein the third location is on a torso of the subject's body;
a fourth transducer adapted to be located at a fourth location of the subject's body, wherein the fourth location is on the torso of the subject's body;
a voltage generator adapted to provide a first voltage to the first transducer, a second voltage to the second transducer, a third voltage to the third transducer, and a fourth voltage to the fourth transducer; and
a controller coupled to the voltage generator, wherein the controller is adapted to instruct the voltage generator to induce a first alternating electric field between at least part of the first transducer and at least part of the second transducer and a second alternating electric field between at least part of the third transducer and at least part of the fourth transducer.

9. The system of claim 8, wherein the third location is under a left armpit of the subject, and the fourth location is under the right armpit of the subject.

10. The system of claim 8, the third location is on the front of the thorax of the subject's body, and the fourth location is under an armpit of the subject, wherein the third location is not overlapping with the first location.

11. The system of claim 8, wherein the third location is on the back of the thorax of the subject's body, and the fourth location is under an armpit of the subject, wherein the third location is not overlapping with the second location.

12. The system of claim 8, when the first and second alternating electric fields are induced, the single lung has a higher average electric field intensity than another lung.

13. The system of claim 8, wherein the at least one electrode element of the first, the second, the third, or the fourth transducer comprises at least one ceramic disk that is adapted to generate an alternating electric field.

14. The system of claim 8, wherein the at least one electrode element of the first, the second, the third, or the fourth transducer comprises a polymer film that is adapted to generate an alternating field.

15. A method of applying tumor treating fields to a subject's body, the method comprising:
locating a first transducer at a first location of the subject's body, wherein the first location is a front of a thorax of the subject's body;
locating a second transducer at a second location of the subject's body, wherein the second location is a back of the thorax of the subject's body, wherein a single lung of the subject is located between the first transducer and the second transducer;
locating a third transducer at a third location of the subject's body, wherein the third location is on a torso of the subject's body;
locating a fourth transducer at a fourth location of the subject's body, wherein the fourth location is on the torso of the subject's body;
inducing a first alternating electric field between at least part of the first transducer and at least part of the second transducer; and
inducing a second alternating electric field between at least part of the third transducer and at least part of the fourth transducer.

16. The method of claim 15, wherein the third location is under a left armpit of the subject and the fourth location is under the right armpit of the subject.

17. The method of claim 15, the third location is on the front of the thorax of the subject's body, and the fourth location is under an armpit of the subject, wherein the third location is not overlapping with the first location.

18. The method of claim 15, wherein the third location is on the back of the thorax of the subject's body, and the fourth location is under an armpit of the subject, wherein the third location is not overlapping with the second location.

19. The method of claim 15, when the first and second alternating electric fields are induced, the single lung has a higher average electric field intensity than another lung.

20. The method of claim 19, wherein the single lung has an average electric field intensity of at least 1.0 V/cm.
